SEQUENCE LISTING (6 sequences)

SEQ ID NO: 1 - DNA, 20 bp, synthetic construct
  source 1..20: mol_type = other DNA; note = primer-16S-338F
actcctacgggaggcagcag

SEQ ID NO: 2 - DNA, 20 bp, synthetic construct
  source 1..20: mol_type = other DNA; note = primer-16S-806R
ggactachvgggtwtctaat

SEQ ID NO: 3 - DNA, 18 bp, synthetic construct
  source 1..18: mol_type = other DNA; note = primer-18S-524F-10-ext
tgycagccgccgcggtaa

SEQ ID NO: 4 - DNA, 19 bp, synthetic construct
  source 1..19: mol_type = other DNA; note = primer-18S- Arch958R-mod
yccggcgttgavtccaatt

SEQ ID NO: 5 - DNA, 22 bp, synthetic construct
  source 1..22: mol_type = other DNA; note = primer-ITS-1F
cttggtcatttagaggaagtaa

SEQ ID NO: 6 - DNA, 20 bp, synthetic construct
  source 1..20: mol_type = other DNA; note = primer-ITS-2R
gctgcgttcttcatcgatgc

(19) United States
(12) Patent Application Publication
(10) Pub. No.: US 2025/0180535 A1
(43) Pub. Date: Jun. 5, 2025

(54) METHOD FOR EVALUATING ECOLOGICAL EFFECTS OF ANTIBIOTICS BASED ON BACTERIAL-ARCHAEAL-FUNGAL CO-OCCURRENCE NETWORK

(71) Applicants: PEKING UNIVERSITY, Beijing (CN); ECO-ENVIRONMENT MONITORING & SCI-RESEARCH CENTER, Bengbu City (CN)

(72) Inventors: Weiling SUN, Beijing (CN); Moran TANG, Beijing (CN); Qian CHEN, Beijing (CN); Tingting ZHANG, Beijing (CN); Xi CHEN, Beijing (CN)

(21) Appl. No.: 18/816,344

(22) Filed: Aug. 27, 2024

(30) Foreign Application Priority Data
Nov. 30, 2023 (CN) 202311615613.1

(51) Int. Cl. G01N 33/15; C12Q 1/6888; C12Q 1/689; C12Q 1/6895

(52) U.S. Cl. CPC G01N 33/15; C12Q 1/6888; C12Q 1/689; C12Q 1/6895; C12Q 2600/156

(57) ABSTRACT

This application relates to the technical field of evaluating ecological effects of antibiotics, and in particular, to a method for evaluating ecological effects of antibiotics based on a bacterial-archaeal-fungal co-occurrence network. In this application, a bacterial-archaeal-fungal co-occurrence network is constructed, removing correlations between the microorganisms in the same group. Based on antibiotic concentrations in samples, the samples are divided into high and low concentration sample groups. By comparing topological properties of networks and nodes in the bacterial-archaeal-fungal co-occurrence networks under the two groups, it is found that under the high antibiotic concentration condition, the average node degree and graph density between different groups of the microorganisms are higher, and the average clustering coefficient and modularity of the network are lower, indicating increased correlations and tighter associations but fewer clustering modules, lower modular differentiation, and lower niche differentiation for different groups of microorganisms under the high antibiotic concentration condition.

### CROSS REFERENCE TO RELATED APPLICATION

This patent application claims the benefit and priority of Chinese Patent Application No. 202311615613.1, filed with the China National Intellectual Property Administration on Nov. 30, 2023, the disclosure of which is incorporated by reference herein in its entirety as part of the present application.

### REFERENCE TO SEQUENCE LISTING

A computer readable XML file entitled “sequence listing”, that was created on Aug. 19, 2024, with a file size of about 7,249 bytes, contains the sequence listing for this application, has been filed with this application, and is hereby incorporated by reference in its entirety.

### TECHNICAL FIELD

The present disclosure relates to the technical field of evaluating ecological effects of antibiotics, and in particular, to a method for evaluating ecological effects of antibiotics based on a bacterial-archaeal-fungal co-occurrence network.

### BACKGROUND

Antibiotics, as a class of secondary metabolites produced by microorganisms and chemically synthesized or semi-synthesized analogous compounds, are important antibacterial agents against bacterial infections. In recent years, various antibiotics such as tetracyclines, macrolides, and quinolones have been successively discovered and widely used in clinical medicine for humans and animals. However, the continuous release of antibiotics leads to their widespread presence in the natural environment. This not only causes pollution to the natural environment but also affects the microbial community structure in the environment, providing unprecedented selective pressure for the evolution and spread of resistant microorganisms. Therefore, understanding the impact of antibiotics on the microorganisms in the natural environment and their mechanisms of action is essential.

In the natural environment, interactions between different groups of microorganisms (bacteria, archaea, and fungi) play an important role in shaping their community structure. The interaction network among microbial populations determines the overall composition, stability, and biodiversity of microbial ecosystems by promoting competition, cooperation, and communication, and further shapes their ecological functions. Microbial co-occurrence networks, which are constructed based on the correlation of microbial taxonomic co-occurrence matrices, are a common statistical analysis method used to study microbial co-occurrence patterns. In microbiology and ecology, microbial co-occurrence networks help us understand patterns of coexistence and exclusion between different microbial species. This method can extract intuitive relationships from complex microbial community structure datasets. Currently, the impact of antibiotics on microbial co-occurrence patterns in natural or artificial systems has only considered the effects of antibiotics on interactions within the same group of microorganisms (such as bacteria), with little research on the effects of antibiotics on co-occurrence patterns between different groups of microorganisms.

Document 1 (CN109785898A) in the prior art discloses a method for evaluating environmental risks of contaminants based on microbial networks. In the construction of networks and evaluation of environmental risks of contaminants, the main steps include: 1) constructing a microbial interaction network based on dominant microbes and key environmental parameters; and 2) evaluating the stability of environmental micro-ecosystems through changes in proportions of negative and positive correlations and average node degree of network in the microbial coexistence relationship. A decrease in the negative correlation proportion and a decrease in the network degree indicate an increased environmental risk of contaminants. The drawback of document 1 in the prior art is that it only explores the evaluation of environmental risks of contaminants (including total nitrogen and phosphorus nutrients, metals zinc and copper, as well as organic contaminants polycyclic aromatic hydrocarbons and halogenated hydrocarbons) based on the proportion of bacteria in the network and average node degree of the network, without considering the impact of antibiotics as emerging contaminants on microorganisms. It also overlooks the impacts of contaminants on the co-occurrence relationships between different groups of microorganisms, i.e., cross-group species, and does not mention the impact of contaminants on the topological properties of different groups of microorganisms in the co-occurrence networks.

Document 2 (CN114707786A) in the prior art discloses a method for evaluating the health of river ecosystems based on a collinear network, where for two groups of microorganisms: bacteria, and archaea in river water and sediment, key indicator species are identified using a co-occurrence network method, to determine alternative parameters. Based on this, the Barbour method and Pearson correlation analysis are used for indicator selection to determine evaluation criteria for river health. Document 2 in the prior art only combines indicators such as the correlation coefficients and node degree of microorganisms in the co-occurrence network for screening to evaluate the health status of rivers, without addressing the impact of contaminants, especially antibiotics, on co-occurrence networks of different groups of microorganisms.

### SUMMARY

In view of this, the present disclosure provides a method for evaluating ecological effects of antibiotics based on a bacterial-archaeal-fungal co-occurrence network. The method provided by the present disclosure can better demonstrate the co-occurrence of different groups of microorganisms, facilitating the visual display of differences in co-occurrence between different groups of the microorganisms under different antibiotic concentration conditions.

To achieve the above objective, the present disclosure provides the following technical solutions.

The present disclosure provides a method for evaluating ecological effects of antibiotics based on a bacterial-archaeal-fungal co-occurrence network, including the following steps:
collecting samples from different sampling sites in a target area, and obtaining antibiotic concentration data as well as community composition and abundance data of microorganisms from the samples, where the microorganisms include bacteria, archaea, and fungi;
calculating a detection frequency of the microorganism in the samples according to formula I, retaining the community composition and abundance data of the microorganism with the detection frequency greater than or equal to 10%, where the detection frequency equals the number of samples in which the microorganism is detected/total number of samples×100% formula I;
separately summing all antibiotic concentrations obtained from each sample, to obtain total detected antibiotic concentration of each sample;
arranging the total detected antibiotic concentrations in ascending order and calculating a cumulative frequency of the total detected antibiotic concentration of each sample according to formula II;
cumulative frequency equals the number of samples with the total detected antibiotic concentrations less than or equal to the total detected antibiotic concentration of a specific sample/total number of samples×100% formula II;
plotting an antibiotic concentration cumulative frequency curve with the total detected antibiotic concentration of each sample as an x-axis and the cumulative frequency corresponding to the total detected antibiotic concentration of each sample as a y-axis, where the total detected antibiotic concentration corresponding to a cumulative frequency of 50% is used as a threshold, samples with the total detected antibiotic concentrations less than or equal to the threshold are considered as low antibiotic concentration samples, while samples with the total detected antibiotic concentrations higher than the threshold are considered as high antibiotic concentration samples;
conducting correlation network analysis separately on the microorganisms in the low antibiotic concentration samples and the high antibiotic concentration samples based on the retained community composition and abundance data of the microorganisms; removing correlations between the microorganisms in the same group and retaining co-occurrence relationships between different groups of the microorganisms; constructing bacterial-archaeal-fungal co-occurrence networks for the high antibiotic concentration samples and the low antibiotic concentration samples respectively, and obtaining topological property parameters of the co-occurrence networks and topological property parameters of nodes, where the topological property parameters of the co-occurrence networks include average node degree, graph density, modularity, and average clustering coefficient, while the topological property parameters of the nodes include degree, transitivity, and betweenness centrality; and
comparing the topological property parameters of the co-occurrence networks and the topological property parameters of the nodes between the high antibiotic concentration samples and the low antibiotic concentration samples to evaluate impact of different antibiotic concentrations on different groups of microorganisms, where a higher average node degree and graph density indicate a closer species correlation between groups; a higher average clustering coefficient and modularity represent higher node aggregation and more clustering modules, indicating a higher degree of niche differentiation; higher degree, transitivity, and betweenness centrality indicate a closer species correlation between groups and a higher microbial correlation between groups.

Optionally, the community composition and abundance data of the bacteria, archaea, and fungi is obtained from the samples by conducting 16S, 18S, and ITS amplicon sequencing on the samples.

Optionally, parameters for the correlation network analysis include microbial data with correlation coefficients greater than or equal to 0.7 and p values ≤0.01.

Preferably, the number of sampling sites for the samples is greater than or equal to 16.

Preferably, the antibiotic concentration data is obtained from the samples by using liquid chromatography-mass spectrometry.

Preferably, the antibiotics include sulfonamides, quinolones, β-lactams, tetracyclines, macrolides, polyethers, and lincomycins.

Preferably, the samples are one or more selected from the group consisting of water samples, soil samples, and sediment samples.

### Beneficial Effects:

The present disclosure constructs a bacterial-archaeal-fungal co-occurrence network, removes correlations between the microorganisms in the same group, and retains only co-occurrence relationships between different groups of the microorganisms, thereby identifying the co-occurrence relationships between different groups of the microorganisms. In the present disclosure, based on antibiotic concentrations in samples, the samples are divided into high and low concentration sample groups. By comparing topological properties of networks and nodes in the bacterial-archaeal-fungal co-occurrence networks under the two groups, it is found that under the high antibiotic concentration condition, the average node degree and graph density between different groups of the microorganisms are higher, and the average clustering coefficient and modularity of the network are lower, indicating increased correlations and tighter associations but fewer clustering modules, lower modular differentiation, and lower niche differentiation for different groups of microorganisms under the high antibiotic concentration condition. This has important value in evaluating the impact of antibiotics on the degree of interrelation and ecological niche differentiation between different groups of microorganisms.

### BRIEF DESCRIPTION OF THE DRAWINGS

FIG. 1 is a flowchart of a method for evaluating ecological effects of antibiotics based on a bacterial-archaeal-fungal co-occurrence network according to the present disclosure;

FIG. 2 is a cumulative frequency graph of total detected antibiotic concentrations in samples;

FIGS. 3A-3B are diagrams of bacterial-archaeal-fungal co-occurrence networks after removal of correlations between the microorganisms in the same group and retention of only co-occurrence relationships between different groups of the microorganisms under high and low antibiotic concentration conditions;

FIGS. 4A-4C show a comparison of network topological properties between the bacterial-archaeal-fungal co-occurrence networks after removal of correlations between the microorganisms in the same group and retention of only co-occurrence relationships between different groups of the microorganisms under high and low antibiotic concentration conditions; and

FIGS. 5A-5C show a comparison of node topological properties between the bacterial-archaeal-fungal co-occurrence networks after removal of correlations between the microorganisms in the same group and retention of only co-occurrence relationships between different groups of the microorganisms under high and low antibiotic concentration conditions.

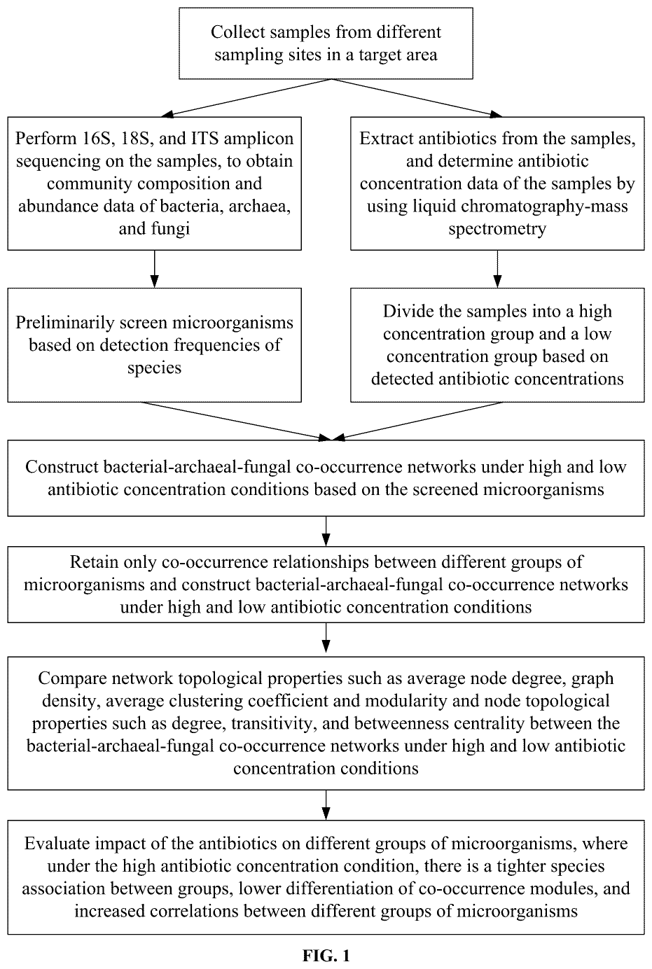

### DETAILED DESCRIPTION OF THE EMBODIMENTS

The present disclosure provides a method for evaluating ecological effects of antibiotics based on a bacterial-archaeal-fungal co-occurrence network, including the following steps:
collecting samples from different points in a target area, and obtaining antibiotic concentration data as well as community composition and abundance data of microorganisms from the samples, where the microorganisms include bacteria, archaea, and fungi;
calculating a detection frequency of the microorganism in the samples according to formula I, retaining the community composition and abundance data of the microorganism with the detection frequency greater than or equal to 10%, where the detection frequency equals the number of samples in which the microorganism is detected/total number of samples×100% formula I;
separately summing all antibiotic concentrations obtained from each sample, to obtain a total detected antibiotic concentration of each sample;
arranging the total detected antibiotic concentrations in ascending order and calculating a cumulative frequency of the total detected antibiotic concentration of each sample according to formula II;
cumulative frequency equals the number of samples with the total detected antibiotic concentrations less than or equal to the total detected antibiotic concentration of a specific sample/total number of samples×100% formula II;
plotting an antibiotic concentration cumulative frequency curve with the total detected antibiotic concentration of each sample as an x-axis and the cumulative frequency corresponding to the total detected antibiotic concentration of each sample as a y-axis, where the total detected antibiotic concentration corresponding to a cumulative frequency of 50% is used as a threshold, samples with the total detected antibiotic concentrations less than or equal to the threshold are considered as low antibiotic concentration samples, while samples with the total detected antibiotic concentrations higher than the threshold are considered as high antibiotic concentration samples;
conducting correlation network analysis separately on the microorganisms in the low antibiotic concentration samples and the high antibiotic concentration samples based on the retained community composition and abundance data of the microorganisms; removing correlations between the microorganisms in the same group and retaining co-occurrence relationships between different groups of the microorganisms; constructing bacterial-archaeal-fungal co-occurrence networks for the high antibiotic concentration samples and the low antibiotic concentration samples respectively, and obtaining topological property parameters of the co-occurrence networks and topological property parameters of nodes, where the topological property parameters of the co-occurrence networks include average node degree, graph density, modularity, and average clustering coefficient, while the topological property parameters of the nodes include degree, transitivity, and betweenness centrality;
comparing the topological property parameters of the co-occurrence networks and the topological property parameters of the nodes between the high antibiotic concentration samples and the low antibiotic concentration samples to evaluate impact of different antibiotic concentrations on different groups of microorganisms, where a higher average node degree and graph density indicate a closer species correlation between groups; a higher average clustering coefficient and modularity represent higher node aggregation and more clustering modules, indicating a higher degree of niche differentiation; higher node degree, transitivity, and betweenness centrality indicate a closer species correlation between groups and a higher microbial correlation between groups.

In the present disclosure, samples are collected from different sampling sites in a target area. In the present disclosure, the number of sampling sites for the samples is preferably greater than or equal to 16, and more preferably greater than or equal to 66; the samples are one or more selected from the group consisting of water samples, soil samples, and sediment samples.

When the samples are water samples, the present disclosure preferably filter the water samples to obtain test samples. In the present disclosure, the filtration is preferably to filter the water samples by using a 0.22 μm filter membrane, and store the filter membrane after the filtration at −80° C. For soil or sediment samples, the collected samples are preferably stored in aluminum boxes at −20° C. for freezing to obtain test samples.

After obtaining the test samples, the present disclosure acquires antibiotic concentration data as well as community composition and abundance data of bacteria, archaea, and fungi in the test samples.

In the present disclosure, for water samples, a preferred method for obtaining antibiotic concentration data in the test sample includes: filtering the water sample with a 0.7 μm glass filter membrane, and adding 100 μL of internal standard solution (with a concentration of 500 ppb) and 0.5 g of Na₂EDTA to every 2 L; after complete dissolution of Na₂EDTA, performing solid-phase extraction to extract and enrich antibiotics, followed by washing, nitrogen purging, and redissolution in a constant volume.

In the present disclosure, for sediment or soil samples, a preferred method for obtaining antibiotic concentration data in the test sample includes: freeze-drying and grinding the sediment or soil sample, sieving through a 2-mm metal sieve, weighing 5.0 g of the sample into a 50-mL glass centrifuge tube, adding 5 mL of internal standard solution (with a concentration of 10 ppb), sealing the tube with a sealing film and storing the tube in a refrigerator at 4° C. for a week; taking out the tube after a week, removing the sealing film, and placing the tube in a fume hood for 36 hours to dry naturally; then performing extraction: using a citric acid buffer solution (0.1 M, pH=3) mixed with acetonitrile in a 1:1 ratio for extraction, followed by centrifugation to obtain the supernatant of the extraction liquid; repeating the extraction process three times; diluting the obtained supernatant with deionized water to 500 mL, adding 0.125 g of Na₂EDTA, and after complete dissolution, performing solid-phase extraction to extract and enrich antibiotics, followed by washing, nitrogen purging, and redissolution in a constant volume.

In the present disclosure, a preferred method for detecting antibiotic concentration data in the samples includes: determining concentrations of antibiotics in a sample using liquid chromatography-mass spectrometry, where the antibiotics preferably include: sulfonamides, quinolones, β-lactams, tetracyclines, macrolides, polyethers, and lincomycins, and more preferably 22 sulfonamides (sulfanilamide, sulfacetamide, sulfachloropyridazine, sulfadiazine, sulfadimethoxine, sulfadoxine, sulfaguanidine, sulfamerazine, sulfametoxydiazine, sulfadimethoxine, sulfamethythiadiazole, sulfamethoxazole, sulfamethoxypyridazine, sulfamonomethoxine, sulfamethazole, sulfaphenazole, sulfapyridine, sulfaquinoxaline, sulfathiazole, sulfadimethoxine, sulfisoxazole, and trimethoprim), 16 quinolones (ciprofloxacin, dafloxacin, difloxacin, enoxacin, enrofloxacin, fleroxacin, flumequine, lomefloxacin, nalidixic acid, norfloxacin, ofloxacin, obifloxacin, oxolinic acid, pefloxacin, sarafloxacin, and sparfloxacin), 15 β-lactams (amoxicillin, ampicillin, cefadroxil, cefapirin, cefazolin, cefotaxime, cefalexin, cefradine, cloxacillin, deacetyl cefotaxime, dicloxacillin, nafcillin, oxacillin, penicillin G, and penicillin V), 13 tetracyclines (4-epianhydrochlortetracycline, 4-epianhydrotetracycline, 4-epichlortetracycline, 4-epioxytetracycline, 4-epitetracycline, anhydrochlorotetracycline, anhydrotetracycline, chlorotetracycline, demethylchlortetracycline, doxycycline, isotretinoin, hygromycin, and tetracycline), 10 macrolides (anhydroerythromycin, azithromycin, clarithromycin, erythromycin, sabinomycin, leucomycin, roxithromycin, spiramycin, tilmicosin, and tylosin), 5 polyethers (lasalocid, maduramicin, monensin, nigericin, and salicylomycin) and 2 lincomycins (clindamycin and lincomycin). A preferred method for obtaining the community composition and abundance data of the bacteria, archaea, and fungi from the samples include conducting 16S, 18S, and ITS amplicon sequencing on the samples.

After the community composition and abundance data of the bacteria, archaea, and fungi is obtained from the samples, a detection frequency of the microorganism in the samples are calculated according to formula I, and the community composition and abundance data of the microorganism with the detection frequency greater than or equal to 10% is retained.

The detection frequency equals the number of samples in which the microorganism is detected/total number of samples×100% formula I.

After the antibiotic concentration data is obtained from the samples, the present disclosure further includes: separately summing all antibiotic concentrations obtained from each sample, to obtain a total detected antibiotic concentration of each sample; and arranging the total detected antibiotic concentrations in ascending order and calculating a cumulative frequency of the total detected antibiotic concentration of each sample according to formula II:

Cumulative frequency equals the number of samples with the total detected antibiotic concentrations less than or equal to the total detected antibiotic concentration of a specific sample/total number of samples×100% formula II.

An antibiotic concentration cumulative frequency curve is plotted with the total detected antibiotic concentration of each sample as an x-axis and the cumulative frequency corresponding to the total detected antibiotic concentration of each sample as a y-axis, where the total detected antibiotic concentration corresponding to a cumulative frequency of 50% is used as a threshold, samples with the total detected antibiotic concentrations less than or equal to the threshold are considered as low antibiotic concentration samples, while samples with the total detected antibiotic concentrations higher than the threshold are considered as high antibiotic concentration samples.

In the present disclosure, correlation network analysis is separately conducted on microorganisms under two antibiotic concentration conditions based on the retained community composition and abundance data of the microorganisms as well as the samples in the high and low concentration groups, removing correlations between the microorganisms in the same group and retaining co-occurrence relationships between different groups of the microorganisms to construct bacterial-archaeal-fungal co-occurrence networks for the high antibiotic concentration samples and the low antibiotic concentration samples respectively. Topological property parameters of the co-occurrence networks and topological property parameters of nodes are obtained. In the present disclosure, preferably, parameters for the correlation network analysis include microbial data with correlation coefficients greater than or equal to 0.7 and p values ≤0.01; the topological property parameters of the co-occurrence networks include average node degree, graph density, modularity, and average clustering coefficient, while the topological property parameters of the nodes include degree, transitivity, and betweenness centrality;

In the present disclosure, for the microorganisms in the samples under high and low antibiotic concentration conditions, Spearman correlation network analysis is preferably conducted based on the MbioAssy 1.0 package in the R environment, removing correlations between the microorganisms in the same group and retaining only co-occurrence relationships between different groups of the microorganisms to construct co-occurrence networks of bacteria, archaea, and fungi under high and low antibiotic concentrations.

The present disclosure preferably uses Gephi (v. 0.9.2) to obtain network topological properties such as average node degree, graph density, modularity, and average clustering coefficient in the co-occurrence networks. For classification information of bacterial, archaeal, and fungal species in different samples under two antibiotic concentration conditions, the topological properties of nodes in the microbial co-occurrence networks, including degree, transitivity, and betweenness centrality, are obtained using the igraph package in the R environment.

In the present disclosure, the impact of antibiotics on different groups of microorganisms is evaluated based on the topological property parameters of the co-occurrence networks and the topological property parameters of the nodes. From the embodiments, it is evident that under the high antibiotic concentration condition, there is a tighter species correlation between groups, lower differentiation of co-occurrence modules, and increased correlations between different groups of the microorganisms.

The technical solution provided by the present disclosure will be described in detail below with reference to the accompanying drawings and embodiments, but they should not be construed as limiting the claimed scope of the present disclosure.

### Example 1

Water samples were collected from 66 sampling sites, with 50 liters of water extracted from each sampling site and filtered using a 0.22 μm sterile filter membrane. The filtered membranes were stored at −80° C. and DNA extraction was carried out separately. Sequencing was performed using 16S, 18S, and ITS amplicons, where primers for amplification have the following sequences:

16S-338F:
SEQ ID NO. 1
5′-ACTCCTACGGGAGGCAGCAG-3′,;
16S-806R:
SEQ ID NO. 2
5′-GG ACTACHVGGGTWTCTAAT-3′,;
18S-524F-10-ext:
SEQ ID NO. 3
5′-TGYCAGCCGCCGCGGTAA-3′,;
18S-Arch958R-mod:
SEQ ID NO. 4
5′-YCCGGCGTTGAVTCCAATT-3′,;
ITS-1F:
SEQ ID NO. 5
5′-CTTGGTCATTTAGAGGAAGTAA-3′,;
ITS-2R:
SEQ ID NO. 6
5′-GCTGCGTTCTTCATCGATGC-3′,.

After purification of PCR products, PE 250 on-sequencer sequencing was performed by using Illumina Miseq 2×250 bp; original raw data was filtered to remove linkers and low-quality bases; USEARCH 8.0 was used to extract clean data from the original data; after a mosaic sequence was removed by using UPARSE, operational taxonomic units (OTUs) of the water samples from 66 sampling points were classified according to a similarity of 97%, and a representative sequence of each OTU cluster was obtained; the representative sequence was compared with a corresponding database by using BLAST software, and species classification information was annotated, that is, species information of the samples was counted at each classification level (domain, phyla, class, order, family, genus, and species), to obtain species classification information of bacteria, archaea, and fungi.

The OTU data reflecting the relative abundance of species obtained from the sequencing was pre-processed to calculate species detection frequencies of all microorganisms (bacteria, archaea, and fungi) across all the samples (species the detection frequency equals number of samples in which the microorganism is detected/total number of samples×100%), and species with detection frequencies greater than or equal to 10% were retained.

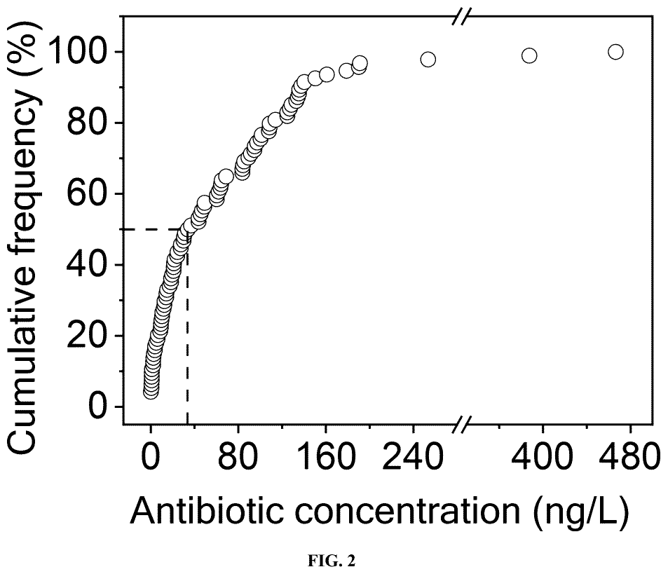

Approximately 4.5 liters of water samples were collected simultaneously from each sampling site in the target area, and the samples were stored in clean brown glass bottles and transported to the laboratory within 24 hours at 4° C. The samples were quickly filtered to remove particulate matter, and then 100 μL of internal standard solution (antibiotic standard with deuterium or C13 substitution, having a concentration of 500 ppb) and 0.5 g of Na₂EDTA were added to every 2 liters of the filtered water sample. After complete dissolution of Na₂EDTA, solid-phase extraction was performed to extract and enrich antibiotics, followed by washing, nitrogen purging, and redissolution in a constant volume. Liquid chromatography-mass spectrometry was used to determine antibiotic concentrations in the samples. Total detected antibiotic concentrations in different samples were obtained, occurrence frequencies of the total detected concentrations where calculated, and an antibiotic concentration cumulative frequency curve was plotted. Based on the number of samples and the distribution pattern of antibiotic concentration frequencies, a low antibiotic concentration group (undetected to 33.8 ng/L) and a high antibiotic concentration (33.8 to 466 ng/L) group of the water samples in this area were determined (as shown in FIG. 2).

Species detection frequencies of all microorganisms (bacteria, archaea, and fungi) across all the samples (species the detection frequency equals number of samples in which the microorganism is detected/total number of samples×100%), and species with detection frequencies greater than or equal to 10% were retained. Under the low antibiotic concentration condition, a total of 7,905 species were obtained through screening, including 5,792 bacteria, 1,284 archaea, and 829 fungi. Under the high antibiotic concentration condition, a total of 7,637 species were obtained through screening, including 5,624 bacteria, 1,195 archaea, and 818 fungi. Compared to the low antibiotic concentration condition, fewer species of the three groups of microorganisms were detected under the high antibiotic concentration condition.

Species classification information for bacteria, archaea, and fungi in the samples under two antibiotic concentration conditions was subjected to Spearman correlation network analysis using the MbioAssy 1.0 package in the R environment to detect co-occurrence patterns of bacteria, archaea, and fungi (retaining bacteria, archaea, and fungi data with a correlation coefficient greater than or equal to 0.7 and p ≤0.01). Further screening was made on the co-occurrence relationships of bacteria, archaea, and fungi under the two antibiotic concentration conditions, removing intra-group autocorrelations among microorganisms and retaining only co-occurrence relationships between different groups of the microorganisms. Under the low antibiotic concentration condition, a total of 809 species showed inter-group correlations, including 475 bacteria, 297 archaea, and 37 fungi. Under the high antibiotic concentration condition, a total of 1,138 species showed inter-group correlations, including 709 bacteria, 384 archaea, and 45 fungi. More species showing inter-group correlations were observed under the high antibiotic concentration condition compared to the low antibiotic concentration condition.

Additionally, under the low antibiotic concentration condition, there were 1,224 edges of co-occurrence between bacteria and archaea, 53 edges of co-occurrence between bacteria and fungi, and 13 edges of co-occurrence between fungi and archaea, totaling 1,290 inter-group correlation edges. Under the high antibiotic concentration condition, there were a total of 2,986 edges of co-occurrence between bacteria and archaea, 87 edges of co-occurrence between bacteria and fungi, and 28 edges of co-occurrence between fungi and archaea, totaling 3,101 inter-group correlation edges, the number of which is greater than that under the low concentration condition.

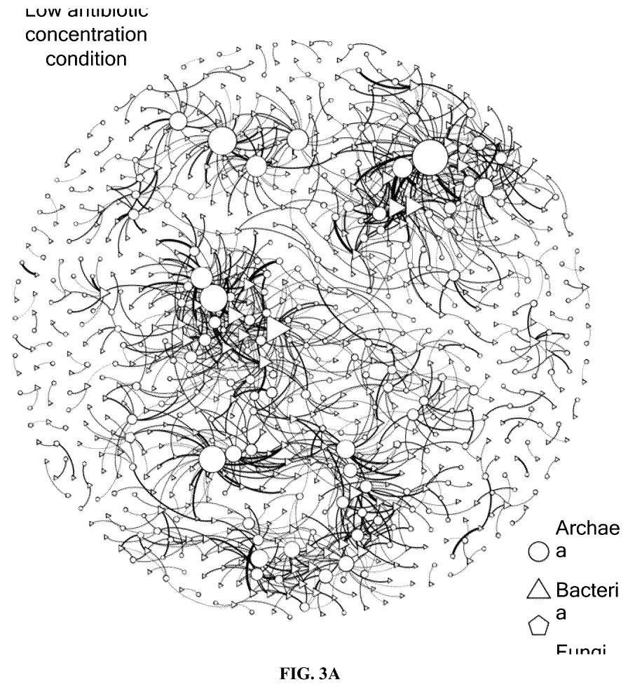

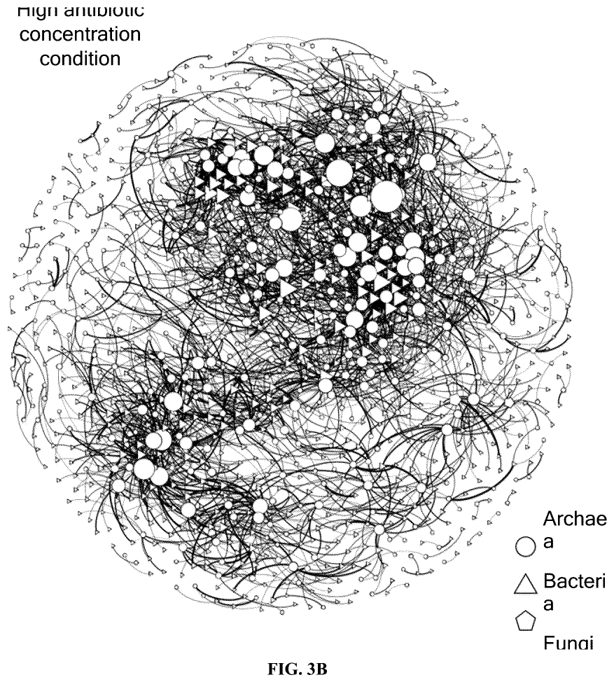

Based on the characteristic data of edges and nodes in the co-occurrence networks for different groups of organisms under high and low concentrations, Gephi (v. 0.9.2) was used to visualize the co-occurrence networks (as shown in FIGS. 3A-3B), and Gephi (v. 0.9.2) was used to obtain the topological properties data of the microbial co-occurrence networks (Table 1).

TABLE 1
Topological properties of microbial co-occurrence networks
under high and low antibiotic concentration conditions

| | Average | | | Average |
|---|---|---|---|---|
| | node | Graph | | clustering |
| Group | degree | density | Modularity | coefficient |
| Low | 3.189 | 0.004 | 0.816 | 0.012 |
| concentration | | | | |
| High | 5.450 | 0.005 | 0.651 | 0.009 |
| concentration | | | | |

The results indicate that under the high antibiotic concentration condition, after removal of autocorrelations between the microorganisms in the same group and retention of only co-occurrence relationships between different groups of the microorganisms, the average node degree and graph density of the co-occurrence network increased, indicating a tighter correlation between species from different groups under the high antibiotic concentration condition. Furthermore, under the high antibiotic concentration condition, the inter-group co-occurrence network had lower modularity and a smaller average clustering coefficient, indicating fewer clustering modules, lower module differentiation, and lower ecological niche differentiation between species from different groups under the high antibiotic concentration condition.

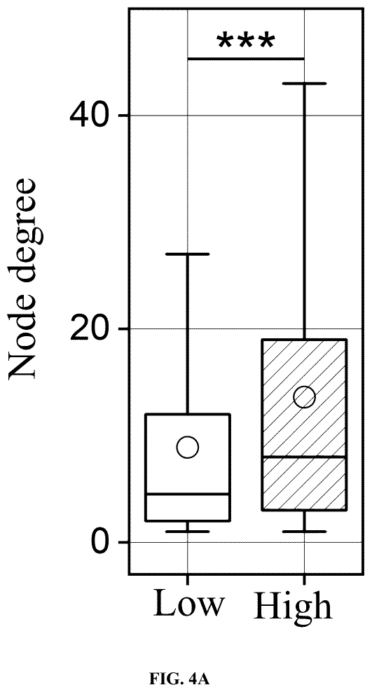

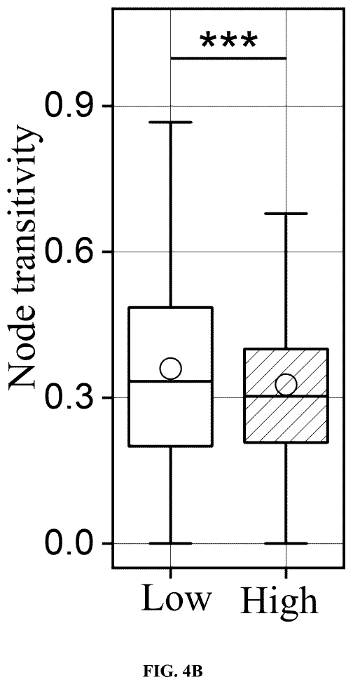

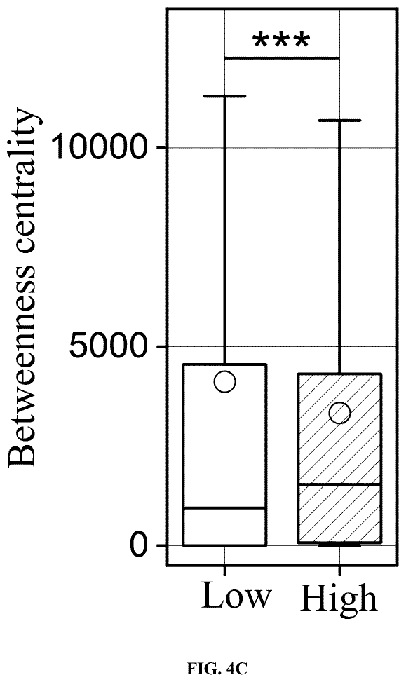

For the classification information of bacterial, archaeal, and fungal species in different samples under two antibiotic concentration conditions, the topological properties of nodes in the microbial co-occurrence networks (including degree, transitivity, and betweenness centrality) were obtained using the induced_subgraph function (igraph package) in the R environment. The results are shown in FIGS. 4A-4C. The results indicate that under the high antibiotic concentration condition, the species degree is significantly higher than that under the low concentration condition, while the transitivity and betweenness centrality of nodes are significantly lower than those under the low concentration condition, further indicating a tighter correlation between species from different groups under the high antibiotic concentration condition, with lower differentiation of co-occurrence modules and increased correlations between different groups of the microorganisms.

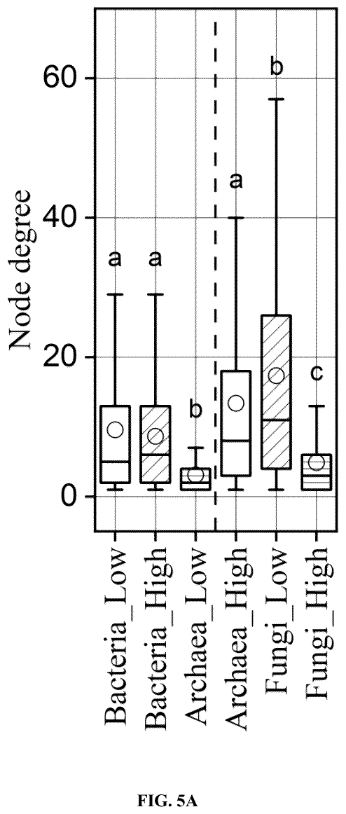

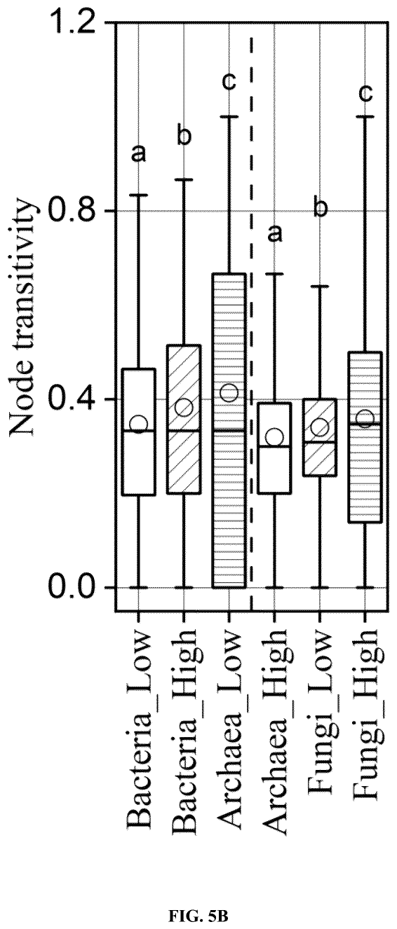

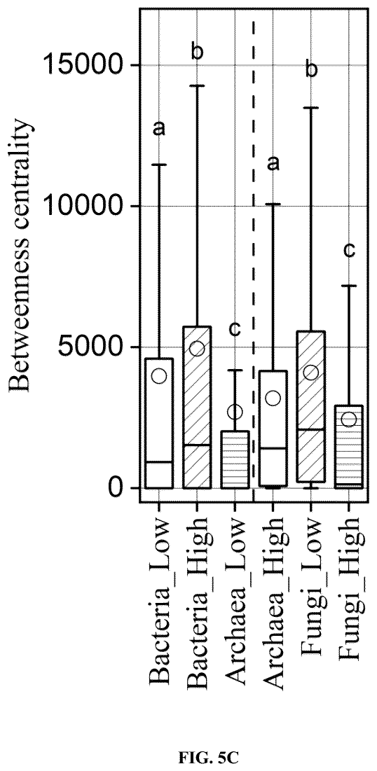

The topological properties of bacteria, archaea, and fungi in the inter-group co-occurrence networks under the low and high antibiotic concentration conditions are shown in FIGS. 5A-5C. The results show that under the low antibiotic concentration condition, the node degree of bacteria and archaea is significantly higher than that of fungi (Mann-Whitney U test: p<0.05); however, under the high antibiotic concentration condition, the node degree of archaea is significantly higher than that of bacteria, and the node degree of bacteria is significantly higher than that of fungi, showing connectivity characteristics inconsistent with those under the low antibiotic concentration condition. Furthermore, the results indicate that archaea have significantly higher betweenness centrality compared to bacteria and fungi under both the low and high antibiotic concentration conditions (FIGS. 5A-5C), suggesting that archaea play a significant role in inter-group associations, especially under the high antibiotic concentration condition.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for evaluating ecological effects of antibiotics based on a bacterial-archaeal-fungal co-occurrence network, comprising the following steps:
collecting samples from different sampling sites in a target area, and obtaining antibiotic concentration data as well as community composition and abundance data of microorganisms from the samples, wherein the microorganisms comprise bacteria, archaea, and fungi;
calculating a detection frequency of the microorganism in the samples according to formula I, retaining the community composition and abundance data of the microorganism with the detection frequency greater than or equal to 10%, wherein the detection frequency equals the number of samples in which the microorganism is detected/total number of samples×100% formula I;
separately summing all antibiotic concentrations obtained from each sample, to obtain a total detected antibiotic concentration of each sample;
arranging the total detected antibiotic concentrations in ascending order and calculating a cumulative frequency of the total detected antibiotic concentration of each sample according to formula II;
cumulative frequency equals the number of samples with the total detected antibiotic concentrations less than or equal to the total detected antibiotic concentration of a specific sample/total number of samples×100% formula II;
plotting an antibiotic concentration cumulative frequency curve with the total detected antibiotic concentration of each sample as an x-axis and the cumulative frequency corresponding to the total detected antibiotic concentration of each sample as a y-axis, wherein the total detected antibiotic concentration corresponding to a cumulative frequency of 50% is used as a threshold, samples with the total detected antibiotic concentrations less than or equal to the threshold are considered as low antibiotic concentration samples, while samples with the total detected antibiotic concentrations higher than the threshold are considered as high antibiotic concentration samples;
conducting correlation network analysis separately on the microorganisms in the low antibiotic concentration samples and the high antibiotic concentration samples based on the retained community composition and abundance data of the microorganisms; removing correlations between the microorganisms in the same group and retaining co-occurrence relationships between different groups of the microorganisms; constructing bacterial-archaeal-fungal co-occurrence networks for the high antibiotic concentration samples and the low antibiotic concentration samples respectively, and obtaining topological property parameters of the co-occurrence networks and topological property parameters of nodes, wherein the topological property parameters of the co-occurrence networks comprise average node degree, graph density, modularity, and average clustering coefficient, and the topological property parameters of the nodes comprise degree, transitivity, and betweenness centrality; and
comparing the topological property parameters of the co-occurrence networks and the topological property parameters of the nodes between the high antibiotic concentration samples and the low antibiotic concentration samples to evaluate impact of different antibiotic concentrations on different groups of microorganisms, wherein a higher average node degree and graph density indicate a closer species correlation between groups; a higher average clustering coefficient and modularity represent higher node aggregation and more clustering modules, indicating a higher degree of niche differentiation; higher node degree, transitivity, and betweenness centrality indicate a closer species correlation between groups and a higher microbial correlation between groups.

2. The method according to claim 1, wherein the community composition and abundance data of the bacteria, archaea, and fungi are obtained from the samples by conducting 16S, 18S, and ITS amplicon sequencing on the samples.

3. The method according to claim 1, wherein parameters for the correlation network analysis comprise microbial data with correlation coefficients greater than or equal to 0.7 and p values less than or equal to 0.01.

4. The method according to claim 1, wherein the number of sampling sites for the samples is greater than or equal to 16.

5. The method according to claim 1, wherein the antibiotic concentration data is obtained from the samples by using liquid chromatography-mass spectrometry.

6. The method according to claim 1, wherein the antibiotics comprise sulfonamides, quinolones, β-lactams, tetracyclines, macrolides, polyethers, and lincomycins.

7. The method according to claim 1, wherein the samples are one or more selected from the group consisting of water samples, soil samples, and sediment samples.

8. The method according to claim 5, wherein the antibiotics comprise sulfonamides, quinolones, β-lactams, tetracyclines, macrolides, polyethers, and lincomycins.
